(19) United States
(12) Patent Application Publication
(10) Pub. No.: US 2025/0375345 A1
(43) Pub. Date: Dec. 11, 2025

(54) Cryogenic Ball Massage Device

(71) Applicant: Franklin Balentine, ASHEVILLE, NC (US)

(72) Inventor: Franklin Balentine, ASHEVILLE, NC (US)

(21) Appl. No.: 18/734,738

(22) Filed: Jun. 5, 2024

(51) Int. Cl. A61H 15/02; A61F 7/00; A61H 15/00

(52) U.S. Cl. CPC A61H 15/02; A61F 7/00; A61F 2007/0045; A61H 2015/0064; A61H 2201/0111; A61H 2201/0157; A61H 2201/0192; A61H 2201/0214; A61H 2201/1253; A61H 2201/169; A61H 2205/12

(57) ABSTRACT

In some aspects, the present invention discloses a cryogenic massage device provided with a base section supporting a molded cryogenic ball holder extending upwardly from the base. The holder has a sloping side wall and forms at least one trough at the top opening to hold a cryogenic ball, sized such that a portion of the ball protrudes above the holder. A liquid trough is formed between the holder and the base to collect melted liquid. At least one adjustment knob on the base adjusts the position or orientation of the holder relative to the base, either by independently adjusting the inclination or by rotating the holder to adjust the orientation to the user's foot position. A cryogenic ball molder shapes a cryogenic substance into balls fitting the trough. The device may have three troughs and knobs, and the base, holder, and liquid trough can be a single molded part.

### FIELD OF THE DISCLOSURE

The present invention relates to the field of therapeutic massage devices, particularly those utilizing ice for cryotherapy. More specifically, the invention pertains to an adjustable ice massage device that holds and applies ice balls to target areas of the body, providing a customizable and ergonomic solution for localized cold therapy treatments, pain relief, and post-exercise recovery.

### BACKGROUND OF THE INVENTION

Ice therapy, also known as cryotherapy, has been used for centuries as a natural method to alleviate pain, reduce inflammation, and promote healing. The application of cold temperatures to the body constricts blood vessels, reducing blood flow to the treated area, which helps to minimize swelling and discomfort associated with various conditions such as muscle strains, sprains, and arthritis. Additionally, ice therapy can numb nerve endings, providing temporary pain relief and promoting relaxation.

Traditionally, ice therapy has been administered using ice packs, cold compresses, or even bags of frozen vegetables. However, these methods have limitations in terms of their ability to target specific areas and maintain consistent temperature throughout the treatment. Moreover, they can be messy and inconvenient, often requiring a cloth barrier between the ice and skin to prevent discomfort or ice burn.

In recent years, various ice massage devices have been developed to address these issues and provide a more targeted and efficient approach to cold therapy. These devices often feature ergonomic designs that allow for easy handling and application of ice to specific body parts. Some incorporate textured surfaces or rollers to enhance the massage effect and promote deeper penetration of the cold into the tissues.

However, many current ice massage devices still have room for improvement in terms of their adaptability and ease of use. Some devices may not effectively contain the melting ice, leading to a messy and slippery experience. Others may have limited ability to adjust the angle or position of the ice, making it difficult to target certain areas comfortably. Furthermore, some devices require frequent refilling or replacement of ice, which can be inconvenient during longer therapy sessions.

The present invention aims to address these shortcomings by providing an innovative ice massage device that offers enhanced adjustability, ergonomics, and convenience. By incorporating features such as an adjustable ice ball holder, a water collection trough, and a custom ice molder, this device allows for a more customizable and streamlined ice massage experience. The primary object of the invention is to provide an effective and user-friendly tool for targeted cold therapy and massage, ultimately promoting pain relief, relaxation, and overall well-being.

### SUMMARY OF THE INVENTION

The following summary provides an overview of some of the key inventive features of the novel cryogenic massage device with adjustable cryogenic ball holder. This summary is not an extensive overview of the invention and is not intended to identify all key or critical elements of the invention or to delineate the scope of the invention. Its sole purpose is to present some concepts of the invention in a simplified form as a prelude to the more detailed description that is presented later.

In some aspects thereof, the present invention is a cryogenic massage device that offers a novel and efficient way to provide targeted cryotherapy to various body parts, particularly the foot, heel, Achilles tendon, and lower leg. The device comprises a base section that supports a molded cryogenic ball holder, which extends upwardly from the base and features a sloping side wall. The holder forms at least one socket at the top opening, designed to hold a cryogenic ball sized such that a portion of the ball protrudes above the holder for effective massage.

One exemplary aspect of the invention is the adjustable nature of the cryogenic ball holder, which allows users to customize the angle of inclination relative to the base section using at least one adjustment knob. This feature ensures that the device can be adapted to comfortably target different body parts and accommodate individual user preferences.

In another aspect, the invention also incorporates a liquid trough positioned between the cryogenic ball holder and the base section, which collects melted liquid from the cryogenic balls during use. This feature helps maintain a clean and mess-free experience for the user.

Further, the device includes a cryogenic ball molder that shapes a cryogenic substance into balls specifically designed to fit into the socket(s) of the cryogenic ball holder. The invention may include multiple sockets and adjustment knobs for enhanced versatility, and in some embodiments, the base, holder, and liquid trough can be formed as a single molded part for improved durability and ease of manufacturing.

In various aspects, the cryogenic massage device provides a convenient, customizable, and effective solution for targeted cryotherapy, offering users a novel way to alleviate pain, reduce inflammation, and promote healing in various body parts.

### BRIEF DESCRIPTION OF THE DRAWINGS

The accompanying drawings, which are incorporated in and constitute a part of this specification, illustrate embodiments of the invention and, together with the description, serve to explain the principles of the invention. The drawings are not necessarily to scale, emphasis instead being placed upon illustrating the principles of the invention. In the drawings:

FIG. 1 is a perspective view of the cryogenic massage device with adjustable cryogenic ball holder, according to one aspect of the invention.

FIG. 2 is a top plan view of the cryogenic massage device, according to one aspect of the invention.

FIG. 3 is a side view of the cryogenic massage device, according to one aspect of the invention.

FIG. 4 is a cross-sectional view of the cryogenic massage device, illustrating the arrangement of the cryogenic balls, sockets, and water collecting basins.

### DETAILED DESCRIPTION OF THE INVENTION

The following detailed description refers to the accompanying drawings, which illustrate specific embodiments of the invention. Other embodiments having different structures and operations do not depart from the scope of the present invention. The same reference numbers may be used in the drawings and the following description to refer to the same or like parts.

As used herein, the terms “comprising,” “including,” “containing,” “characterized by,” and grammatical equivalents thereof are inclusive or open-ended and do not exclude additional, unrecited elements or method steps, unless otherwise stated. Other than in the operating examples, or where otherwise indicated, all numbers expressing measurements, dimensions, and so forth used in the specification and claims are to be understood as being modified in all instances by the term “about,” meaning within a reasonable range of the indicated value. The terms “a” and “an” refer to one or more of the elements described, whereas the term “plurality” refers to two or more of the elements described, unless the context clearly indicates otherwise.

The cryogenic massage device with adjustable cryogenic ball holder described herein provides novel solutions for efficient and customizable cryotherapy experiences, particularly for targeted massage of various body parts such as the foot, heel, Achilles tendon, and lower leg. The invention incorporates an adjustable cryogenic ball holder, a liquid collection trough, and a cryogenic ball molder, enabling adaptable massage angles, increased user comfort, and convenient use. The following detailed description, along with the accompanying drawings, provides a comprehensive understanding of the various embodiments and aspects of the invention.

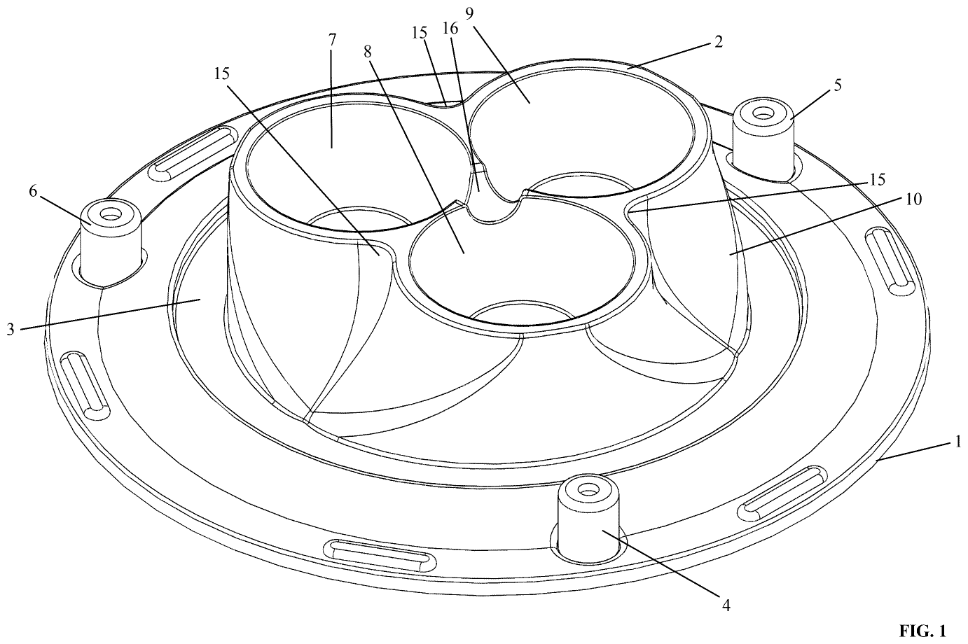

The embodiment of FIG. 1 illustrates a perspective view of the cryogenic massage device with an adjustable cryogenic ball holder, according to one aspect of the invention. The device comprises a base (1) that provides support and stability to the entire assembly. Extending upwardly from the base (1) is a molded cryogenic ball holder (2), which features a sloping side wall (10) that extends from the top opening towards the base (1).

The cryogenic ball holder (2) forms multiple sockets (7, 8, 9) at the top opening, each configured to hold at least one cryogenic ball. These troughs are sized and shaped such that a portion of each cryogenic ball protrudes above the top opening of the holder (2), allowing for effective massage of various body parts.

Positioned between the sloping side wall of the cryogenic ball holder (2) and the base (1) is a water trough (3). This trough is designed to collect water from the melting cryogenic balls during use, helping to maintain a clean and mess-free experience for the user.

In an exemplary non-limiting aspect, the adjustable nature of the cryogenic ball holder (2) is facilitated by three adjustment knobs (4, 5, 6) provided on the base (1). These knobs can be independently adjusted to retract into or extend downward from the base (1), allowing users to customize the angle of inclination of the cryogenic ball holder (2) relative to the base (1). This feature ensures that the device can be adapted to comfortably target different body parts, such as the foot, heel, Achilles tendon, and lower leg, while accommodating individual user preferences.

In an alternative embodiment of the cryogenic massage device, the control knobs (4, 5, 6) are designed to function differently than in the previously described embodiment. Instead of tilting the stand by lifting one side off the ground, the control knobs in this embodiment are used to rotate the stand and adjust the orientation of the cryogenic balls (11, 12, 13) and the stand itself relative to the user's foot position.

This functionality can be compared to a steering wheel knob, where the user can easily manipulate the position of the stand by turning the knobs. The knobs are strategically placed on the perimeter of the base (1), allowing the user to control the rotation of the stand effortlessly with their non-treatment foot or hand.

In addition to the rotational movement, this embodiment also allows the user to push the stand further away or pull it closer to them. This feature is particularly useful when the user is sitting in a chair with the cryogenic massage device placed on the floor in front of them. During the treatment session, the user may need to occasionally adjust the position of the stand to target different areas of their foot, heel, Achilles tendon, or lower leg.

By pushing the stand forward, the user can effectively apply more pressure to the desired area, while pulling the stand closer allows for a gentler massage or a change in the targeted region. The side-to-side rotation of the stand, controlled by the knobs, enables the user to fine-tune the angle and position of the cryogenic balls to achieve optimal coverage and effectiveness.

Between the sockets (7, 8, 9) of the molded cryogenic ball holder (2), there are recesses (15) that serve a specific purpose in the design of the cryogenic massage device. These recesses are strategically placed areas where material has been removed to provide additional clearance, allowing for a maximum range of motion during use.

In the exemplary illustration shown in FIG. 1, there are three recesses (15) located on the outside of the molded cryogenic ball holder (2), positioned between each cryogenic ball socket (7, 8, 9). These recesses are carefully designed to accommodate the natural movement of the user's foot during the massage session.

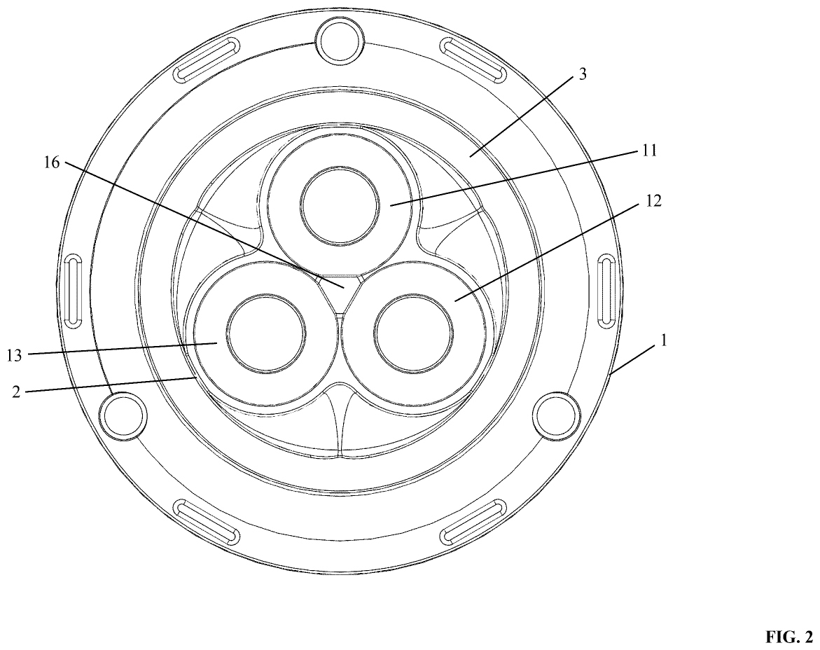

As the user angles their foot downward and positions it between the cryogenic balls (11, 12, 13) as shown in FIG. 2, the bottom of the mid-foot generally slides by the outside area between the balls. The recesses (15) provide the necessary space for this movement, ensuring that the user can comfortably and effectively target the desired areas of their foot, heel, Achilles tendon, or lower leg.

The crux or valley between two cryogenic balls is another area where the user's foot may naturally settle during the massage session. The recesses (15) in these areas allow for a greater range of motion, enabling the user to apply pressure and manipulate their foot position as needed for optimal massage coverage and effectiveness.

By incorporating these strategically placed recesses (15) into the design of the molded cryogenic ball holder (2), the cryogenic massage device offers enhanced comfort, flexibility, and adaptability to accommodate various foot sizes and massage techniques.

On the other hand, the FIG. 2 is an exemplary top plan view of the cryogenic massage device, offering a further illustration of some key components and their arrangement. The illustration features a sturdy base (1) that provides stability and support for the entire assembly. Positioned on the base (1) is a water trough (3), which is formed at the bottom portion of the cryogenic ball holder and situated between the holder's sloping side wall and the base. This trough is designed to collect water from the melting cryogenic balls during use, preventing spills and messes.

The exemplary non-limiting cryogenic massage device in this illustration is designed to accommodate up to three cryogenic balls (11, 12, 13) of suitable diameter, typically ranging from 40-80 mm. The device preferably comes with a dedicated ice mold for creating cryogenic balls of the appropriate size. In other aspects, the mold may be detachable for placing in a freezer. In other aspects, a cone to cover the mold may be provided in addition to form an enclosure when placing in a fridge to shape ice in desired shape. The arrangement of the cryogenic balls within the holder allows users to effectively apply ice massage treatment to various parts of the foot, heel, Achilles tendon, and lower leg.

In some aspects, the device has advantages due to its hands-free design, which allows users to sit comfortably while applying the cryogenic massage. The cryogenic balls are held in an advantageous position for treatment, and as the heat from the body melts the ice, the water slides along the slope of the holder into the water trough (3), preventing any spills or messes during use.

To further enhance user comfort and customization, the device features three control knobs (not shown in this view) positioned on the perimeter of the base (1). These knobs allow users to easily adjust the position of the cryogenic balls using either their non-treatment foot or hand, ensuring a tailored and efficient massage experience.

In addition to the recesses (15) located on the outside of the molded cryogenic ball holder (2), there is also a recess positioned in the middle, between the sockets (7, 8, 9). This central recess serves two main purposes, both of which contribute to the overall functionality and user-friendliness of the cryogenic massage device.

Firstly, the central recess improves the range of motion, especially when the user is utilizing just two cryogenic balls (11, 12) for their massage session. When using two balls, the user is most often targeting the Achilles tendon area. In this scenario, the lower or back portion of the heel naturally wants to drop down into the center cutout area provided by the recess. This design feature allows for a more comfortable and effective massage experience, as the user can easily position their heel in the desired location without any obstruction.

Secondly, the center cutout serves as a convenient way for the user to lift the cryogenic balls back out of the stand when needed. Due to the slippery nature of the ice, if the sockets (7, 8, 9) were to completely surround each ball, the user would have difficulty getting underneath the balls to lift them out. In such a case, the user would be required to tip the stand and dump out the balls, along with any ice water that had collected in the water trough (3) below. However, with the center cut away, the user can easily insert a finger under the ‘equator’ of the cryogenic ball and apply upward pressure to lift it out of the socket.

This feature is particularly useful when the user wants to transition from using three balls to two, or when they need to flip the balls after the top portion has melted, and the bottoms are more full and round. The central recess provides a convenient and practical solution for manipulating the cryogenic balls as needed throughout the massage session.

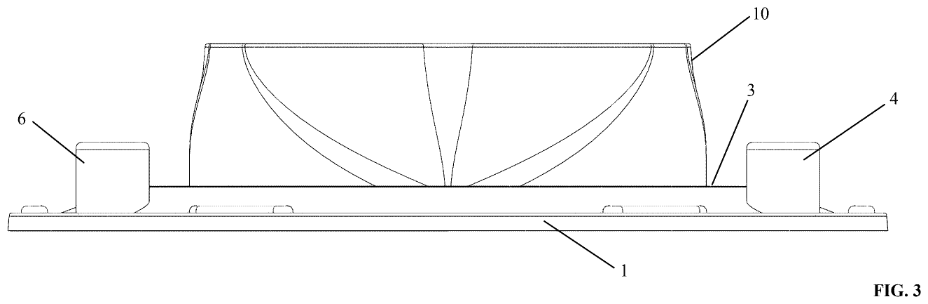

The exemplary embodiment of FIG. 3 presents a side view of the cryogenic massage device, offering a detailed look at its profile and the arrangement of its components. The device features a sturdy base (1) that serves as the foundation for the entire assembly, providing stability and support during use.

Extending upwardly from the base (1) is the cryogenic ball holder, which features a sloping side wall (10). This sloping design allows for the proper positioning of the cryogenic balls within the holder, ensuring that a portion of each ball protrudes above the top opening for effective massage.

Located at the bottom portion of the cryogenic ball holder and positioned between the holder's sloping side wall (10) and the base (1) is a water trough (3). This trough is designed to collect water from the melting cryogenic balls during use, preventing spills and messes. As the heat from the user's body melts the ice, the water travels along the sloping side wall (10) and is efficiently collected in the water trough (3).

The side view also showcases two of the three control knobs (4, 6) that are positioned on the perimeter of the base (1). These knobs allow users to easily adjust the angle of inclination of the cryogenic ball holder relative to the base, ensuring a customized and comfortable massage experience. By turning the knobs, users can retract or extend the knobs downward from the base, effectively changing the angle of the cryogenic ball holder to target specific areas of the foot, heel, Achilles tendon, or lower leg.

The side view in FIG. 3 highlights the device's ergonomic design and the thoughtful placement of its components. The sloping side wall (10) of the cryogenic ball holder, combined with the water trough (3) and adjustable control knobs (4, 6), demonstrate the device's ability to provide efficient and targeted cryotherapy while maintaining a user-friendly experience.

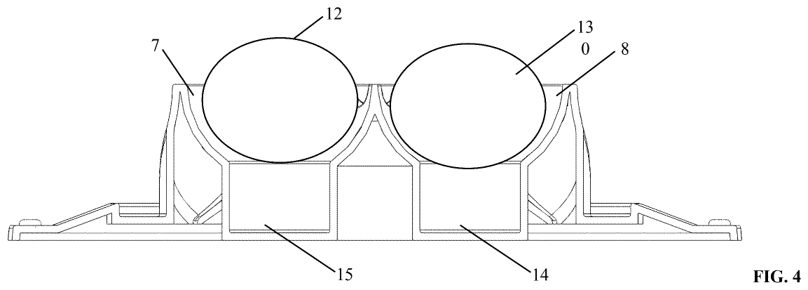

Reference is now made to FIG. 4, which presents a cross-sectional view of the cryogenic massage device, providing a detailed look at the internal structure and arrangement of its key components. The device features multiple sockets (7, 8) formed within the molded cryogenic ball holder (2), each designed to hold a cryogenic ball (12, 13).

The sockets (7, 8) are sized and shaped to accommodate the cryogenic balls (12, 13) securely, with a portion of each ball protruding above the top opening of the holder (2). This protrusion allows for effective contact between the cryogenic balls and the user's foot, heel, Achilles tendon, or lower leg during the massage session.

Positioned at the bottom of each socket (7, 8) are water collecting basins (15, 14), which serve as melt basins for the cryogenic balls (12, 13). As the cryogenic balls gradually melt during use, the water collecting basins (15, 14) capture and contain the melted liquid, preventing it from spilling or leaking onto the base section (1) or the surrounding area.

The water collecting basins (15, 14) are strategically placed directly beneath each cryogenic ball socket (7, 8), ensuring that the melted liquid is efficiently collected and contained. This design feature helps to maintain a clean and mess-free experience for the user throughout the cryotherapy session.

Various aspects of the invention illustrate a water collection system strategically formed at the bottom portion of the cryogenic ball holder. This water collection system is designed to efficiently collect and contain water resulting from the melting of the cryogenic balls during use. In one embodiment, the water collection system comprises a water trough positioned between the sloping side wall of the cryogenic ball holder and the base section. This water trough serves to capture and hold the melted water, preventing it from spilling onto the base or surrounding area. In another embodiment, the water collection system includes a plurality of water collecting basins, each positioned beneath a corresponding trough of the at least one trough formed in the cryogenic ball holder. These water collecting basins act as individual melt basins, catching and retaining the water from each melting cryogenic ball separately. The incorporation of the water collection system, whether as a single water trough or multiple water collecting basins, ensures a mess-free and convenient user experience throughout the cryotherapy massage session.

In one exemplary embodiment, the cryogenic massage device may be used by athletes or fitness enthusiasts seeking targeted relief for foot, heel, or lower leg pain. After an intense training session or competition, the user can sit comfortably and place their affected foot on the cryogenic balls within the device's holder. By adjusting the control knobs, the user can customize the angle of inclination to focus the massage on specific areas, such as the plantar fascia, heel, or Achilles tendon. The cold temperature of the cryogenic balls helps to reduce inflammation, numb pain, and promote recovery. As the ice melts during the massage session, the water is collected in the trough, ensuring a mess-free experience. This targeted cryotherapy can be incorporated into the user's post-workout routine to help prevent and manage common overuse injuries.

In another exemplary embodiment, the cryogenic massage device may be used by individuals suffering from chronic foot conditions, such as plantar fasciitis or heel spurs. The user can place their affected foot on the cryogenic balls and adjust the angle of inclination to target the specific area of discomfort. The cold therapy helps to reduce pain and inflammation associated with these conditions, while the massage action promotes circulation and relaxation. The user can engage in this targeted cryotherapy session for the recommended duration, as advised by their healthcare provider. The hands-free design of the device allows the user to sit comfortably and read, work, or watch television during the massage session, making it a convenient and effective way to manage chronic foot pain in the comfort of their own home.

### BEST MODE OF CARRYING OUT THE INVENTION

The preferred best mode of carrying out the invention involves the use of a cryogenic massage device with an adjustable cryogenic ball holder. To begin, the user fills the provided ice mold with water and places it in the freezer to create cryogenic balls of the appropriate size, typically ranging from 40-80 mm in diameter. Once the balls are frozen, the user places them into the sockets at the top opening of the cryogenic ball holder.

The user then sits comfortably and positions their foot, heel, Achilles tendon, or lower leg on the cryogenic balls. The adjustable nature of the device allows the user to customize the angle of inclination of the cryogenic ball holder by turning the control knobs located on the perimeter of the base. This ensures that the massage targets the desired area effectively.

As the heat from the user's body melts the cryogenic balls, the water travels along the sloping side wall of the holder and collects in the water trough positioned between the holder and the base. This design prevents spills and messes during use. The user can enjoy a hands-free, targeted cryotherapy session for the desired duration, adjusting the angle of inclination as needed for optimal comfort and effectiveness.

### BENEFITS OF THE INVENTION

The cryogenic massage device with an adjustable cryogenic ball holder offers numerous benefits for individuals seeking targeted cold therapy and massage for various foot, heel, and lower leg conditions. One of the primary advantages of this device is its adjustable design, which allows users to customize the angle of inclination of the cryogenic ball holder to target specific areas effectively. This feature ensures that users can focus the massage on the exact location of their pain or discomfort, whether it be the plantar fascia, heel, Achilles tendon, or other parts of the foot and lower leg. The device's hands-free operation enables users to sit comfortably during the massage session, making it convenient and easy to use in various settings, such as at home or in a sports therapy clinic.

Another significant benefit of the cryogenic massage device is its ability to provide efficient and mess-free cryotherapy sessions. The device's innovative design incorporates a water trough positioned between the cryogenic ball holder and the base, which collects the water from the melting ice during use. This feature prevents spills and messes, ensuring a clean and hassle-free experience for the user. Additionally, the device comes with a dedicated ice mold, allowing users to create cryogenic balls of the appropriate size for optimal massage coverage and effectiveness. The combination of targeted cold therapy and massage action helps to reduce pain, inflammation, and swelling associated with various foot and lower leg conditions, promoting faster recovery and overall well-being.

### INDUSTRIAL APPLICATION

The cryogenic massage device with an adjustable cryogenic ball holder has valuable industrial applications in the fields of sports medicine, physical therapy, and occupational health. The device can be utilized by professional sports teams, fitness centers, and rehabilitation clinics to provide targeted cold therapy and massage for athletes and patients recovering from foot, heel, and lower leg injuries. Furthermore, the device can be incorporated into workplace wellness programs, particularly in industries where employees are prone to lower extremity fatigue and discomfort, such as manufacturing, construction, and healthcare. By offering on-site cryotherapy sessions, employers can help reduce the incidence of work-related musculoskeletal disorders, improve employee comfort, and boost overall productivity.

What is claimed is:

1. An cryogenic massage device comprising:
a base section providing support to the device;
a molded cryogenic ball holder extending upwardly from the base section, the cryogenic ball holder having a substantially sloping side wall extending from a top opening towards the base section, the cryogenic ball holder forming at least one socket opening at the top configured to hold at least one cryogenic ball, wherein the at least one socket is sized and shaped such that a portion of the at least one cryogenic ball protrudes above the socket opening when the at least one cryogenic ball is disposed within the at least one socket;
a water collection system formed at a bottom portion of the cryogenic ball holder, the water collection system configured to collect water melted from cryogenic balls;
at least one adjustment knob provided on the base section, the at least one adjustment knob configured to adjust a position or orientation of the cryogenic ball holder relative to the base section or a user's foot position; and
a cryogenic molder configured to shape cryogenic into cryogenic balls sized to fit into the at least one trough of the cryogenic ball holder.

2. The cryogenic massage device of claim 1, wherein the at least one adjustment knob is configured to independently adjust an angle of inclination of the cryogenic ball holder relative to the base section.

3. The cryogenic massage device of claim 1, wherein the at least one adjustment knob is configured to rotate the cryogenic ball holder and adjust an orientation of the at least one cryogenic ball relative to a user's foot position.

4. The cryogenic massage device of claim 1, wherein the cryogenic ball holder comprises three sockets, each trough configured to hold one cryogenic ball.

5. The cryogenic massage device of claim 1, wherein the at least one adjustment knob comprises three adjustment knobs.

6. The cryogenic massage device of claim 1, wherein the at least one adjustment knob is configured to be adjusted to retract into or extend downward from the base section.

7. The cryogenic massage device of claim 1, wherein the base section, the cryogenic ball holder, and the water trough are formed as a single molded part.

8. The cryogenic massage device of claim 1, wherein the at least one adjustment knob is configured to tilt the base section relative to the cryogenic ball holder to adjust the angle of inclination of the cryogenic ball holder.

9. The cryogenic massage device of claim 1, wherein the cryogenic ball holder is configured to position the at least one cryogenic ball to massage a user's lower leg.

10. The cryogenic massage device of claim 1, wherein the water collection system comprises a water trough positioned between the cryogenic ball holder sloping side wall and the base section.

11. The cryogenic massage device of claim 1, wherein the water collection system comprises a plurality of water collecting basins, each water collecting basin positioned beneath a corresponding trough of the at least one trough formed in the cryogenic ball holder.

12. A method of using an cryogenic massage device, the method comprising:
providing an cryogenic massage device comprising:
a base section providing support to the device;
a molded cryogenic ball holder extending upwardly from the base section, the cryogenic ball holder having a substantially sloping side wall extending from a top opening towards the base section, the cryogenic ball holder forming at least one socket at the top opening;
a water collection system formed at a bottom portion of the cryogenic ball holder, the water collection system configured to collect water melted from cryogenic balls;
at least one adjustment knob provided on the base section; and
a cryogenic molder;
shaping cryogenic into at least one cryogenic ball using the cryogenic molder, wherein the at least one cryogenic ball is sized to fit into the at least one socket of the cryogenic ball holder;
placing the at least one cryogenic ball into the at least one socket such that a portion of the at least one cryogenic ball protrudes above the top opening of the cryogenic ball holder; and
adjusting a position or orientation of the cryogenic ball holder relative to the base section or a user's foot position using the at least one adjustment knob.

13. The method of claim 12, wherein the water collection system comprises a water trough positioned between the cryogenic ball holder sloping side wall and the base section.

14. The method of claim 12, wherein the water collection system comprises a plurality of water collecting basins, each water collecting basin positioned beneath a corresponding trough of the at least one trough formed in the cryogenic ball holder.

15. The method of claim 12, wherein adjusting the position or orientation of the cryogenic ball holder comprises adjusting an angle of inclination of the cryogenic ball holder relative to the base section using the at least one adjustment knob.

16. The method of claim 12, wherein adjusting the position or orientation of the cryogenic ball holder comprises rotating the cryogenic ball holder and adjusting an orientation of the at least one cryogenic ball relative to a user's foot position using the at least one adjustment knob.

17. The method of claim 12, wherein adjusting the angle of inclination of the cryogenic ball holder comprises adjusting the at least one adjustment knob to retract into or extend downward from the base section.

18. The method of claim 12, wherein providing the cryogenic massage device comprises providing the cryogenic massage device wherein the base section, the cryogenic ball holder, and the water trough are formed as a single molded part.

19. The method of claim 12, wherein adjusting the angle of inclination of the cryogenic ball holder comprises tilting the base section relative to the cryogenic ball holder using the at least one adjustment knob.

20. The method of claim 12, further comprising massaging a user's lower leg with the at least one cryogenic ball by positioning the cryogenic ball holder accordingly.
